(19) United States
(12) Patent Application Publication
(10) Pub. No.: US 2005/0008695 A1
(43) Pub. Date: Jan. 13, 2005

(54) Compositions and methods for delivering a biologically active agent

(75) Inventors: Paul Ashton, Boston, MA (US); Jianbing Chen, Belmont, MA (US); Hong Guo, Belmont, MA (US); Grazyna Cynkowska, Brookline, MA (US); Tadeusz Cynkowski, Brookline, MA (US)

Correspondence Address: ROPES & GRAY LLP, ONE INTERNATIONAL PLACE, BOSTON, MA, 02110-2624, US

(73) Assignee: Control Delivery Systems, Inc., Watertown, MA (US)

(21) Appl. No.: 10/850,957

(22) Filed: May 21, 2004

Related U.S. Application Data
(60) Provisional application No. 60/479,827, filed on Jun. 18, 2003.
(60) Provisional application No. 60/472,706, filed on May 21, 2003.

(51) Int. Cl. A61K 9/20

(52) U.S. Cl. 424/464

(57) ABSTRACT

The invention relates to codrugs having improved properties, methods for preparing and administering them, and methods of formulating and administering the codrugs as pharmaceutical preparations. In certain embodiments, the codrugs can be locally administered to deliver the constituent biologically active compound in a sustained-release fashion, reducing systemic concentrations of the biologically active compound.

### RELATED APPLICATIONS

This application claims the benefit of U.S. provisional application 60/472706, filed May 21, 2003, and 60/479827, filed Jun. 18, 2003, the specifications of which are incorporated by reference herein.

### BACKGROUND OF THE INVENTION

Different drug compounds have different physiochemical properties as a result of their unique chemical structures. Some drug compounds are highly soluble in water, while others are poorly soluble in water. Some drug compounds are unstable in certain environments, such as the acidic environment of the stomach, and/or have undesirable effects in certain environments, such as causing stomach ulcers when ingested. Modem medicine's success and economic efficiency in treating physiological conditions are often limited by the physical and biological characteristics of available therapeutics. For example, drugs that are effective in the treatment of disease may not readily be formulated in a physiologically acceptable delivery device suitable for treating that disease, and hence may not readily be delivered to specific physiological sites in need of the drug. In other cases, the number of synthetic steps involved in producing therapeutically effective drugs limits the cost effectiveness of such drugs. The availability of some drugs is decreased due to short shelf-lives. In certain instances, the formulations comprising a drug may undermine the drug's treatment utility or make the formulation process complicated and expensive. Achieving sustained release of some drugs requires complex formulations involving multiple processing steps. Accordingly, new drug compounds that alleviate one or more of these problems without adversely affecting therapeutic efficacy would be advantageous.

### SUMMARY OF THE INVENTION

The present invention provides for drug formulations that improve the production and physiological delivery of pharmaceutically active compounds, preferably the delivery of pharmaceutically active small molecules. In particular, the invention provides codrugs, each having at least two drug moieties covalently linked together where each moiety corresponds to a constituent compound having a biological activity, or a prodrug form thereof. The codrugs have improved properties, as described herein, as compared to the properties of their constituent compounds. The invention also relates to pharmaceutical compositions comprising one or more pharmaceutically acceptable carriers, diluents, adjuvants or excipients in combination with the codrugs.

The invention also provides a method of treating a subject in need of such treatment, comprising administering to a patient in need thereof a therapeutically effective amount of a codrug or a pharmaceutical preparation thereof as described herein, wherein the codrug provides for more effective delivery of the constituent compounds.

### BRIEF DESCRIPTION OF THE DRAWINGS

FIG. 1 shows the release profile of a homocodrug of diclofenac.

FIG. 2 shows the release profile of a diclofenac-ciprofloxacin codrug salt.

FIG. 3 compares the drug release profiles of a solution of a codrug according to the invention and a commercially available eye drop product.

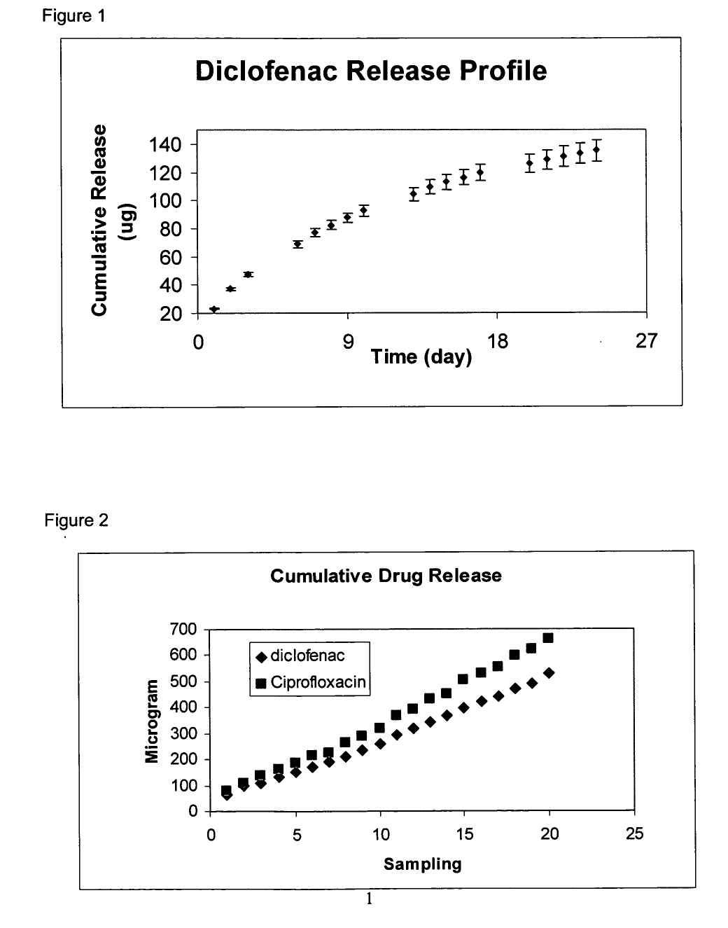

### DETAILED DESCRIPTION OF THE INVENTION

I. Overview

The present invention provides codrugs having improved properties as compared to the properties of their constituent compounds, pharmaceutical compositions comprising the codrugs, and therapeutic methods of using the codrugs.

In some embodiments, the codrugs are stable in solid form, and are sparingly soluble in aqueous solvent, for instance in physiologic fluids or in aqueous solutions at or near at physiologic pH, but preferably rapidly cleave or dissociate to release the constituent compounds when solubilized. As a result, in such embodiments, the parent compound can be released in aqueous solvent in a time-released manner, e.g., controlled primarily by the rate of dissolution.

In preferred embodiments, the codrug is moderately soluble or even highly soluble in aqueous solvent, e.g., in those solutions identified above. The codrugs claimed herein may be in free acid or free base form.

In certain embodiments, a codrug or a prodrug thereof may take the form of a homocodrug, wherein the constituent moieties are the same compound. In such embodiments, the codrug may release, upon cleavage or dissociation, two or more molecules of a single drug compound. In other embodiments, a codrug takes the form of a heterocodrug, wherein the constituent moieties are different compounds. In such embodiments, the codrug may release, upon cleavage or dissociation, molecules of two or more different drug compounds.

In certain other embodiments, the codrug or a prodrug thereof can be delivered in a therapeutically effective dose to a site within a body in need of treatment, thereby enabling delivery of the constituent residues in therapeutically effective dosages to a site within such a body. In still other embodiments, a codrug compound can be delivered at a single time in a single therapeutically effective dose in a controlled manner.

II. Definitions

As used herein, the term “EC₅₀” means the effective concentration of a drug, it being a dose of a drug that produces 50% of its maximum response or effect. In preferred embodiments, the compounds A₁ and A₂ are comparably equipotent when administered to a patient, e.g., both compounds have effective concentrations (EC₅₀'s) for a target receptor or other biological target within an order of magnitude of each other, preferably within a factor of five, or even within a factor of two. In certain embodiments the biological activity of compounds A₁ and A₂ may be the same or different.

The phrase “pharmaceutically acceptable carrier” as used herein means a pharmaceutically acceptable material, composition or vehicle, such as a liquid or solid filter, diluent, adjuvant, excipient, solvent or encapsulating material, involved in carrying or transporting a subject drug from one organ, or portion of the body, to another organ, or portion of the body. Each carrier must be “acceptable” in the sense of being compatible with other ingredients of the formulation and not injurious to the patient. Some examples of materials which can serve as pharmaceutically acceptable carriers include (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. Preferred carriers are non-pyrogenic, i.e., do not substantially elevate the body temperature of a patient receiving the formulation.

The term “codrug” as used herein means a compound, or a prodrug form thereof, comprising a first residue associated with a second residue, wherein both residues, in their unlinked forms (e.g., in the absence of the association), are biologically active. In preferred embodiments, one or both of the residues is a small molecule. The association between said residues is covalent and is either direct or indirect through a linker. The first residue can be the same or different from the second. The codrugs referred to herein may optionally be homocodrugs or heterocodrugs. A “homocodrug,” also termed a “symmetrical codrug,” refers to a codrug that produces, upon cleavage or dissociation, two or more molecules of a single drug, and no other drug molecules, i.e., the homocodrug is composed primarily of two or more residues of a single drug, without incorporating a residue of a second drug. A “heterocodrug,” also termed an “asymmetrical codrug,” refers to a codrug that produces, upon cleavage or dissociation, residues of at least two different drugs.

The term “prodrug” as used herein means a first residue associated with a second small molecule residue, wherein one of the residues is not biologically active. In preferred embodiments, one or both of the residues is a small molecule. In some embodiments, the prodrug may be biologically inactive in its prodrug form. The association between said residues is covalent and can be either direct or indirect through a linker. Prodrugs of biologically active compounds include esters, as well as anhydrides, amides, and carbamates that are hydrolyzed in biological fluids to produce the parent compounds.

The term “covalently linked” as used herein means either a direct covalent bond between two species, or an indirect association where two species not directly bonded but are both covalently bonded to an intermediate linker.

The term “improved in vivo stability” means that a compound decomposes more slowly in vivo than does either or both of the constituent compounds. In preferred embodiments, the codrugs decompose at least 20% more slowly than the constituent compounds, preferably at least 50% more slowly.

The term “substantially pyrogen-free” means a pharmaceutical composition having a pyrogen (e.g., endotoxin) concentration of less than about 0.3 EU/ml, preferably less than about 0.03 EU/ml, or even 0.01 EU/ml. The term also refers to a compound having a pyrogen contaminant (e.g., endotoxin) concentration of less than about 0.3 EU/mg, preferably less than about 0.03 EU/mg, or even 0.01 EU/mg.

The term “moderately soluble,” as used herein to describe solubility of a compound in aqueous solution, refers to a solubility greater than 30 mg/ml but less than 100 mg/ml, preferably greater than about 50 mg/ml and less than 100 mg/ml. A compound that is “sparingly soluble” in aqueous solution has a solubility greater than about 10 mg/ml and less than 30 mg/ml. A compound that is “highly soluble” in aqueous solution has a solubility greater than 100 mg/ml, preferably greater than about 500 mg/ml. A compound that has “low solubility” in aqueous solution has a solubility less than 10 mg/ml, preferably less than 5 mg/ml.

The phrase “protecting group” or “protective group” as used herein means a temporary substituent that protects a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

The term “residue” refers to that part of a compound that remains after the compound is linked, either directly to the other compound by a direct bond or to a divalent linking moiety. For instance, where a residue A₁ comprises a carboxylic acid group that forms a linkage to a second residue A₁ through an amino group to form the compound A₁-A₁, including an amide linkage, the first residue A₁ is the residue of the parent compound that includes all of the parent except for the —OH that forms part of the amide group, while the other includes all of the parent except an H— from the amino group. A person having skill in the art will recognize that this is analogous to “residues” of amino acids in polypeptides and proteins, or to “residues” of ribonucleotides and deoxyribonucleotides in RNA and DNA, respectively.

III. Codrugs and Compositions Thereof

In preferred embodiments according to the present invention, a codrug having moieties A₁ and A₂ may be represented by formula (I):
A₁(—L—A₂)ₙ (I)

wherein:

A₁ is a compound having a biological activity or a prodrug thereof; A₂ is also a compound having a biological activity or a prodrug thereof; A₂ may be the same or different compound than A₁;

L is a linking group selected from a direct bond and a divalent organic linking group; and

n is an integer having a value of from 1 to 4, preferably 1.

Each occurrence of A₁ and/or A₂ in a codrug can feature a different residue of the compound. For example, and without intending to be limiting, where a homocodrug is prepared utilizing A₁ as a hydroxyacid (e.g., comprising two residues of A₁, i.e., A₁=A₂), one occurrence of A₁ might be a residue attached through its hydroxyl group, while the other occurrence of A₁ might be a residue attached through its carboxyl group. In other embodiments, for example, where a heterocodrug is used (i.e., A₂ ≠A₁), the codrug combining A₁ and A₂ may be prepared using A₁ attached through its carboxyl group, while a different codrug combining A₁ and A₂ may be prepared using A₁ attached through its hydroxyl group. Similar variations using different functional groups of A₂ are also contemplated.

In certain embodiments, L may be a direct bond (e.g., the residues of A₁ as described above may be attached directly via an ester bond), or L may be a linking group, such as an amino acid (e.g., linking the two groups via an amide bond and an ester bond). The moieties may be linked, for example, directly or indirectly through an ester, an amide, a carbamate, a carbonate, a cyclic ketal, a thioester, a thioamide, a thiocarbamate, a xanthate, a phosphate ester, etc. In preferred embodiments, the constituent residues may be regenerated by the cleavage of the bond(s) linking the moieties together.

Embodiments using L in formula (I) as a direct bond may be represented by formula (Ia):
A₁(—A₂)ₙ (Ia)
wherein A₁, A₂, and n are as defined above, and may include salts of codrugs represented by formula (Ia).

In some preferred embodiments according to the present invention, compounds are represented by one of the formulae II, Ia, III, and IIIa, below.
A₁—L—A₂ (II)
A₁—A₂ (IIa)
A₁—L—A₂—L—Aₓ (III)
A₁—A₂—Aₓ (IIIa)
wherein each of A₁, A₂ and L are as defined above, although in preferred embodiments L is not absent from the formula. Aₓ may be A₁ or A₂.

In certain embodiments, the codrugs described above can be prepared by first providing a precursor compound, represented in general by one of formulae (IV) through (Va) below:
A*₁—L—A*₂ (IV)
A*₁—A*₂ (IVa)
A*₁—L—A*₂—L—A*ₓ (V)
A*₁—A*₂—A*ₓ (Va)
wherein moiety A*₁ is a precursor of a residue of a parent compound A₁ (as defined above), wherein A*₁ can be converted through a series of reactions, e.g. four or fewer reactions, into residue —A₁; moiety A*₂ is, in preferred embodiments, a precursor of a residue of a parent compound A₂ (as defined above), wherein A*₂ can be converted through a series of reactions, e.g. four or fewer reactions, into residue —A₂. In certain embodiments, A*₂ may be the same as A*₁, thereby forming a homocodrug precursor. A*₂ may be different than A*₁, thereby forming a heterocodrug precursor. A*ₓ may be A*₁ or A*₂. In preferred embodiments, a precursor residue has at least one site that is biologically inactive but is biologically active in the parent compound. For example, and without limitation, where A₁ has a reactive carboxyl group, A*₁ may be formed by converting the carboxyl group to a biologically inactive amide. In still other embodiments, A*₂ need not be a precursor but may be A₂ or a residue thereof.

In certain embodiments, A*₁ and A*₂ may be converted, respectively, to A₁ and A₂ when any of precursor compounds (IV) through (Va) are subjected to reactions. The resulting products are codrug compounds i.e., any of compounds (II) through (IIIa). Such reactions may include the addition of groups or moieties, e.g., acyl, phosphoryl, sulfate, sulfonate, alkyl, amino, amide. Such reactions may also include oxidation, reduction, and the cleavage of individual groups or moieties, e.g., the cleavage of an acetyl group. However, in preferred embodiments where the reaction results in the cleavage of a group, the cleaved group is preferably not a group that would ordinarily be cleaved under physiological conditions. For example, where a precursor compound is converted by cleaving a group, the cleaved group is not a protecting group in preferred embodiments. After conversion of the precursor residues to their respective parent compounds (e.g. A₁ and A₂, respectively), the codrug may be cleaved under physiological conditions to release the parent compounds. In embodiments where A*₂ is not a precursor but is A₂ or a residue thereof, the reactions are preferably applied only to A*₁ and not to A*₂.

Codrugs according to the present invention preferably have improved properties as compared to properties of the constituent compounds from which they are derived. For example, A₁ may decompose more slowly under ambient conditions and/or ordinary storage conditions (i.e., about 25° C.) (and thereby have a longer shelf life) when stored in codrug form according to any of formulae (I) through (IIIa) than when stored in non-codrug form (e.g., as an unlinked compound(s)). In certain embodiments, A₁ in its codrug form has a decomposition rate of at least 1 0% less than its decomposition rate as an unlinked compound in the same formulation at room temperature. In preferred embodiments A₁ in its codrug form has a decomposition rate of at least 25%, preferably even 50% lower than its decomposition rate when existing as an unlinked compound in the same formulation at room temperature. In some embodiments, the decomposition rate of A₂ in its codrug form has a decomposition rate of at least 10% less than its decomposition rate when existing as an unlinked compound in the same formulation at room temperature; in preferred embodiments, A₂ in its codrug form has a decomposition rate of at least 25%, preferably even 50% lower than its decomposition rate when existing as an unlinked compound in the same formulation at room temperature.

In another aspect, a codrug may provide for easier formulation as compared to the formulation of its unlinked constituent compounds. For example, a codrug may be more soluble in a polymeric delivery system. In preferred embodiments, the codrug is at least 10% more soluble in a polymeric delivery system, preferably at least 25% more soluble, or even at least 50% more soluble than at least one of the unlinked constituent compounds.

In other embodiments, the codrugs may be more readily formulated as a powder. In other embodiments, the codrugs may be more readily formulated as a crystalline matrix.

In some embodiments, a codrug may be more readily mixed with a pharmaceutically acceptable carrier, e.g., an excipient. For example, a codrug may be more soluble than at least one of the unlinked compounds in a pharmaceutically acceptable carrier. In preferred embodiments, the codrug's solubility in a pharmaceutically acceptable carrier is at least 10% greater than the solubility of at least one of the unlinked constituent compounds in the carrier. In other embodiments, the codrug's solubility is at least 25% greater, even at least 50% greater in a pharmaceutically acceptable carrier than the solubility of at least one of the unlinked constituent compounds in the carrier.

In still other embodiments, the codrugs may more readily be adapted than the unlinked constituent compounds for use in solid dosage forms, e.g., where a codrug is a solid at room temperature and one or more unlinked constituent compounds are liquids at room temperature. In such embodiments, the constituent compounds may be prepared, stored, and/or delivered with greater convenience and/or efficiency in the codrug form than in the unlinked form.

In certain embodiments, the codrugs according to the present invention have improved in vivo stability. In preferred embodiments, codrugs according to the present invention may provide sustained release of the constituent compounds over an extended period, preferably without the use of a semi-permeable membrane. In certain embodiments, the sustained release occurs over a period of at least 24 hours; preferably, the sustained release occurs over at least 2 days, or even at least one week or at least one month.

In certain embodiments, codrugs according to the present invention have increased solubility at the same temperature in physiological fluids as compared to the solubility of constituent compounds. In preferred embodiments, the codrugs are at least 10% more soluble, preferably at least 25% or even 50% or greater, than the unlinked constituent compounds at the same temperature in physiological fluids.

In certain embodiments, providing one or more drugs as a codrug (e.g., a homocodrug or heterocodrug) promotes the release of the constituent drugs in a sustained fashion over a period of time, e.g., 3 days, 5 days, a week, two weeks, a month, or even six months.

In certain embodiments, codrugs according to the present invention may have increased solubility and/or stability in high pH environments (i.e., pH above 7.4, in certain embodiments above 8.5) as compared to the solubility and/or stability of the unlinked constituent compounds. In preferred embodiments, the codrugs are at least 10% more soluble, preferably at least 25% or even 50% or greater, than the unlinked constituent compounds at the same temperature in high pH environments. In preferred embodiments, the codrugs are at least 15% more stable, preferably at least 25% or even 50% or greater, than the unlinked constituent compounds at the same temperature in high pH environments.

In other embodiments, codrugs according to the present invention may have increased solubility and/or stability in low pH environments (i.e., below pH 7.4, in certain embodiments, below 6.5) as compared to the solubility and/or stability of the unlinked constituent compounds. In preferred embodiments, the codrugs are at least 10% more stable, preferably at least 25% or even 50% or greater, than the unlinked constituent compounds at the same temperature in low pH environments. In certain embodiments, a codrug of any of formulae (I) through (IIIa) may be more easily manufactured when synthesized by the use of precursor compounds of formulae (IV) through (Va) than when synthesized by combining the constituent compounds. For example, a codrug of any of formulae (I) through (IIIa) may be more easily manufactured when synthesized by converting A*₁—L—A*₂ to A₁—L—A₂, than by combining A₁ and A₂ through linking group L.

In preferred embodiments, manufacturing a codrug through precursor compounds (e.g., those described above) requires the use of fewer manipulations than processes that combine the constituent compounds in separate steps. In preferred embodiments, the codrugs can be prepared by simplified processes that facilitate the masking of reactive groups located on the constituent compounds. For example, standard synthetic processes often require the masking of a reactive group (e.g., a hydroxyl group) prior to or in connection with the synthesis. In such cases, the masking is done by subjecting the reactive group to a known reaction (e.g., carboxylating the hydroxyl group) prior to completing the synthesis of the constituent compound. Subsequent to the masking, and preferably in the final stages of the synthesis, the reactive group is unmasked by converting the masking group (e.g., the carboxyl group) back to the reactive group. According to the present invention, the separate steps of masking and unmasking can be eliminated by joining precursor compounds (e.g., A₁* and/or A₂*) either directly or indirectly through the reactive group, (e.g., either directly or indirectly through a hydroxyl group). The result is, for example, an intermediate compound of any of formulae (IV) through (Va), that can then be subjected to further reactions to form codrug compounds, e.g., compounds of any of formulae (I) through (IIIa). The foregoing is illustrated by the following reaction scheme:
Precursor with reactive group (G): G—A*₁ (VI)
Masking reaction: (G—A*₁)+(G—A*₁)→A*₁—G—L—G—A*₁ (VII)
Synthetic step(s): A*₁—G—L—G—A*₁→A₁—G—L—G—A₁ (VIII)

In certain embodiments, multiple precursors with the same or different reactive groups may be combined as illustrated in the following reaction scheme:
Precursors with reactive group (Gₓ): G₁—A*₁, G₂—A*₂ (IX)
Masking reaction: (G₁—A*₁)+(G₂—A*₂) A*₁—G₁—L—G₂—A*₂ (X)
Synthetic step(s): A*₁—G₁—L—G₂—A*₂→A₁—G₁—L—G₂—A₂ (XI)
wherein reactive group G₁ is the same or different than reactive group G₂, and A*₁ is different than A*₂.

In still other embodiments, A*₂ need not be a precursor but may, instead, be a residue of parent compound A₂. An example of such an embodiment is illustrated as follows:
Precursor with reactive group (G₁): G₁—A*₁ (XII)
Masking reaction: (G₁—A*₁)+A₂→A*₁—G₁—L—A₂ (XIII)

Synthetic step(s): A*₁—G₁—L—A₂→A₁—L—A₂ (XIV)

The codrugs possess other advantages when compared to the unlinked constituent compounds. In certain embodiments, the codrugs may dissolve more readily in organic solvent and therefore may be more readily extracted by standard methods than are the unlinked constituent compounds. In preferred embodiments, the codrugs are at least 10% more soluble than the unlinked constituents in organic solvent, preferably at least 25% or even at least 50% more soluble.

In another aspect, drugs prepared in codrug form according to the invention exhibit comparable physiological release profiles to those of the unlinked constituent compounds. In some embodiments, drugs prepared in codrug form according to the invention have release profiles and other pharmacokinetic characteristics that are therapeutically equivalent to the unlinked constituent compounds. In preferred embodiments, compositions comprising the codrugs are substantially pyrogen-free.

In certain embodiments, any or all of compounds A₁, A*₁, A₂, and/or A*₂ (or residues thereof) may be chiral. In some embodiments, the codrugs are substantially enantiomerically pure.

In certain embodiments according to the present invention, at least one of the codrug's constituent compounds is an antineoplastic, an anti-bacterial, a non-steroidal anti-inflammatory (NSAID), a glucocorticoid, or other anti-inflammatory corticosteroid, such as a topical anti-inflammatory steroid, an anti-angiogenesis agent, an alkaloid analgesic, such as an opioid analgesic, an antiviral, such as a nucleoside antiviral or a non-nucleoside antiviral, or other therapeutic compound.

Suitable NSAID compounds include diclofenac, etoldolac, fenoprofen, floctafenine, flurbiprofen, ibuprofen, indoprofen, ketoprofen, ketorolac, lomoxicam, morazone, naproxen, perisoxal, pirprofen, pranoprofen, suprofen, suxibuzone, tropesin, ximoprofen, zaltoprofen, zileuton, and zomepirac, and pharmaceutically acceptable salts, esters, prodrugs and protected forms thereof.

Suitable alkaloid analgesics include desmorphine, dezocine, dihydromorphine, dimepbeptanol, eptazocine, ethylmorphine, glafenine, hydromorphone, isoladol, ketobenidone, p-lactophetide, levorphanol, moptazinol, metazocin, metopon, morphine, nalbuphine, nalmefene, nalorphine, naloxone, norlevorphanol, normorphine, oxmorphone, pentazocine, phenperidine, phenylramidol, tramadol, and viminol, and pharmaceutically acceptable salts, esters, prodrugs and protected forms thereof.

Suitable glucocorticoids include 21-acetoxypregnenolone, aldlometasone, algestone, aincinonide, beclomethasone, betamethasone, budesonide, chloroprednisone, clobetasol, clobetasone, clocortolone, cloprednol, corticosterone, cortisone, cortivazol, deflazacort, desonide, desoximetasone, diflorasone, diflucortolone, difuprednate, enoxolone, fluazacort, flucloronide, flumethasone, flunisolide, fluocinolone acetonide, fluocinonide, flucloronide, flumethasone, flunisolide, fluocortin butyl, fluocortolone, fluorometholone, fluperolone acetate, fluprednisolone, flurandrenolide, fluticasone propionate, hydrocortamate, hydrocortisone, meprednisone, methylprednisolone, paramethasone, prednisolone, prednisolone 21-diethylaminoacetate, fluprednidene acetate, formocortal, loteprednol etabonate, medrysone, mometasone furoate, prednicarbate, prednisolone, prednisolone 25-diethylaminoacetate, prednisolone sodium phosphate, prednisone, prednival, prednylidene, triamcinolone, triamcinolone acetonide, triamcinolone benetonide, and triamncinolone hexacetonide, and pharmaceutically acceptable salts, esters, prodrugs and protected forms thereof.

Other suitable corticosteroids include halcinonide, halbetasol propionate, halometasone, halopredone acetate, isoflupredone, loteprednol etabonate, mazipredone, rimexolone, and tixocortol, and pharmaceutically acceptable salts, esters, prodrugs and protected forms thereof.

Suitable anti-benign prostatic hypertrophy (BPH) drugs include finasteride and osaterone, and pharmaceutically acceptable salts, esters, prodrugs and protected forms thereof.

Suitable antineoplastic compounds include alitretinoin (9-cis-retinoic acid); bleomycins, including bleomycin A; capecitabine (5′-deoxy-5-fluoro-cytidine); carubicin; chlorozotocin, chromomycins, including chromomycin A₃, cladribine; colchicine, cytarabine; daunorubicin; demecolcine, denopterin, docetaxel, doxyifluridine, doxorubicin; dromostanolone, edatrexate, enocitabine, epirubicin, epitiostanol, estramustine; etoposide; floxuridine, fludarabine, formestane, gemcitabine; irinotecan; lentinan, lonidamine, melengestrol, melphalan; menogaril, methotrexate; mitolactol; nogalamycin; nordihydroguaiaretic acid, olivomycins such as olivomycin A, paclitaxel; pentostatin; pirarubicin, plicamycin, porfiromycin, prednimustine, puromycin; ranimustine, ristocetins such as ristocetin A; temozolamide; teniposide; tomudex; topotecan; tubercidin, ubenimax, valrubicin (N-trifluoroacetyladriamycin-14-valerate), vinorelbine, vinblastine, vindesine, vinorelbine, and zorubicin and pharmaceutically acceptable salts, esters, prodrugs and protected forms thereof.

Suitable antibacterial compounds include capreomycins, including capreomycin IA, capreomycin IB, capreomycin IIA and capreomycin IIB; carbomycins, including carbomycin A; carumonam; cefaclor, cefadroxil, cefamandole, cefatrizine, cefazedone, cefazolin, cefbuperazone, cefcapene pivoxil, cefclidin, cefdinir, cefditoren, cefime, ceftamet, cefinenoxime, cefinetzole, cefminox, cefodizime, cefonicid, cefoperazone, ceforanide, cefotaxime, cefotetan, cefotiam, cefoxitin, cefpimizole, cefpiramide, cefpirome, cefprozil, cefroxadine, cefsulodin, ceftazidime, cefteram, ceftezole, ceftibuten, ceftiofur, ceftizoxime, ceftriaxone, cefuroxime, cefuzonam, cephalexin, cephalogycin, cephaloridine, cepholosporin C, cephalothin, cephapirin, cephamycins, such as cephamycin C, cephradine, chlortetracycline; chlarithromycin, clindamycin, clometocillin, clomocycline, cloxacillin, cyclacillin, danofloxacin, demeclocyclin, destomycin A, dicloxacillin, dicloxacillin, dirithromycin, doxycyclin, epicillin, erythromycin A, ethanbutol, fenbenicillin, flomoxef, florfenicol, floxacillin, flumequine, fortimicin A, fortimicin B, forfomycin, foraltadone, fusidic acid, gentamycin, glyconiazide, guamecycline, hetacillin, idarubicin, imipenem, isepamicin, josamycin, kanamycin, leumycins such as leumycin A₁, lincomycin, lomefloxacin, loracarbef, lymecycline, meropenam, metampicillin, methacycline, methicillin, mezlocillin, micronaomicin, midecamycins such as midecamycin A₁, mikamycin, minocycline, mitomycins such as mitomycin C, moxalactam, mupirocin, nafcillin, netilicin, norcardians such as norcardian A, oleandomycin, oxytetracycline, panipenam, pazufloxacin, penamecillin, penicillins such as penicillin G, penicillin N and penicillin O, penillic acid, pentylpenicillin, peplomycin, phenethicillin, pipacyclin, piperacilin, pirlimycin, pivampicillin, pivcefalexin, porfiromycin, propiallin, quinacillin, ribostamycin, rifabutin, rifamide, rifampin, rifamycin SV, rifapentine, rifaximin, ritipenem, rekitamycin, rolitetracycline, rosaramicin, roxithromycin, sancycline, sisomicin, sparfloxacin, spectinomycin, streptozocin, sulbenicillin, sultamicillin, talampicillin, teicoplanin, temocillin, tetracyclin, thostrepton, tiamulin, ticarcillin, tigemonam, tilmicosin, tobramycin, tropospectromycin, trovafloxacin, tylosin, and vancomycin, and pharmaceutically acceptable salts, esters, prodrugs and protected forms thereof.

Suitable linking groups, L, within the scope of embodiments according to the present invention include direct bonds and divalent organic linking groups, including -alkylene-, -alkyl-oxy-alkyl-, -alkyl-amino-alkyl-, -alkyl-thio-alkyl-, -amino-alkyl-amino-, -oxy-alkyl-oxy-, -carbonyl-alkyl-carbonyl-, -carbonylamino-alkyl-alkylcarbonyl-, and -carbonyloxy-alkyl-oxycarbonyl- (wherein each -alkylene- and -alkyl- group independently has 1 to 12 carbon atoms, and where possible may be branched or unbranched).

In some embodiments, the codrugs may be deployed on a stent or other drug delivery device. Such devices include, but are not limited to surgical screws, prosthetic joints, artificial valves, plates, pacemakers, sutures, etc. In certain embodiments, the codrugs are not formulated in a hydrogel.

In certain embodiments of the present invention, the compounds are delivered through a bioerodible drug delivery device capable of delivering one drug or even two or more synergistic drugs over a prolonged period. In preferred embodiments, the device allows delivery of the compounds over a period of at least 3 hours, preferably at least 12 hours, or even 1 day, at least 2 days, or even at least 1 week, 1 month, or 1 year. In certain embodiments, the device is formed of a bioerodible polymer matrix selected from polyanhydride, polylactic acid, polyglycolic acid, polyorthoester, polyalkylcyanoacrylate, and derivatives and copolymers thereof. In other embodiments, the device may be non-bioerodible, for example comprising a non-bioerodible polymer matrix selected from polyurethane, polysilicone, poly(ethylene-co-vinyl acetate), polyvinyl alcohol, and derivatives and copolymers thereof. In preferred embodiments, the non-bioerodible device allows delivery of the compounds over a period of at least 1 day, preferably at least 2 days, or even at least 1 week, 1 month, or 1 year.

For example, but without limitation, U.S. Pat. No. 5,378,475, U.S. Pat. No. 5,773,019, U.S. Pat. No. 5,902,598, U.S. Pat. No. 6,001,386, and U.S. Pat. No. 6,375,972 disclose various embodiments of sustained release drug delivery devices. Such devices may be usefully employed with the systems described herein, and the entire disclosures of those references are incorporated herein by reference.

In another aspect, the invention contemplates administering the codrugs, compositions, and devices discussed herein to a patient. Certain aspects of the improved properties described above facilitate this administration. For example, as generally described above, the codrugs have the advantage that linking the two moieties, e.g., through carbamate, carbonate, ester, or other bonds linking the molecules, decreases the solubility of the codrug relative to one or both of the unlinked constituent compounds in aqueous solutions such as bodily fluids. In some embodiments, the codrugs have a high degree of chemical or enzymatic lability at physiological pH 7.4. A combination of low solubility and high chemical or enzymatic lability at physiologic pH provides that codrugs according to the present invention may be injected at or near the locus of desired therapeutic activity, where they will be released slowly into the surrounding tissue and quickly converted into the active constituent compound upon exposure to physiologic conditions, thereby producing a high local concentration of the constituent compound. Because systemic administration is avoided by this method, the systemic concentrations of the residues may remain low, while the localized concentrations may be maintained within the therapeutic range over a period of time ranging from days to months.

The codrugs may be administered to a patient in need thereof in injectable form, such as in liposomes, liquids, suspensions and microsphere nanoparticles. Preparation of such aqueous solutions, liposomes, emulsions, and suspensions are known to those skilled in the art. See Remington's Pharmaceutical Sciences, 18th Ed., Mack Publishing Co., Easton, Pa., 1990, pp. 1504-1712, incorporated herein by reference. The codrugs may be administered in any art-recognized fashion. For example, oral, rectal, parenteral (subcutaneous, intravenous, intramuscular), intrathecal, transdermal, and other such forms of administration may be employed. Systemic administration may also be employed. Dosage forms include tablets, troches, dispersions, suspensions, solutions, capsules, patches, and the like. In some preferred embodiments according to the invention, one or more compressed pellets of a codrug are implanted into the target tissue, for instance by subcutaneous or intramuscular injection.

Some compositions of the present invention can be produced as suspensions, solutions, elixirs, and aerosols. Codrugs produced according to the invention can also be produced as a therapeutic and can be delivered through a pharmaceutically acceptable carrier. Carriers such as starches, sugars, microcrystalline cellulose, diluents, granulating agents, lubricants, binders, disintegrating agents and the like may be used in the case of oral solid preparations. Oral solid preparations (such as powders, capsules, and tablets) are preferred over the oral liquid preparations. Tablets are the most preferred oral solid preparation. If desired, tablets may be coated by standard aqueous or non-aqueous techniques.

In certain embodiments, codrugs according to the present invention exist as dimers, as exemplified in the attachment. Exemplary compounds can be seen in the attachment.

IV. Exemplification

One of skill in the art may further comprehend the invention by reference to the following non-limiting examples. It will be apparent to one skilled in the art that various modifications, both to starting materials and methods, are encompassed within the invention, and may be adopted without departing from the scope of the invention.
Diclofenac-5-Fluorouracil (5-FU)

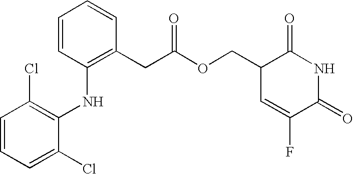

Hydrolysis Rate (half-life t½)

| In Buffer, pH7.4 | In Human Plasma |
|---|---|
| 3.5 hrs | 65 mins |

Conjugation of diclofenac to 5-FU lowers the solubility of 5-FU and promotes synergistic effects, e.g., in the treatment of cancer and other proliferative diseases. This codrug can be used in topical formulations, sustained-release polymeric formulations, and/or as a coating for implants such as stents. For example, this codrug can be provided in the compositions and devices disclosed in U.S. patent application Ser. No. 10/316,137, and PCT Applications WO 02/87586 and WO 03/024455.
Diclofenac-Fluocinolone Acetonide

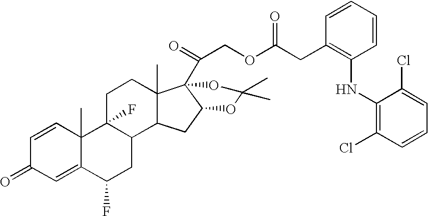

Hydrolysis Rate (half life t½)

| In Buffer, pH7.4 | In Human Plasma |
|---|---|
| 15 hrs | 7 days |

This codrug has a lower solubility under aqueous conditions than either of the constituent drugs, and thus may be suitable for sustained-release formulations, including depot injections and topical formulations, such as those discussed in U.S. patent application Ser. No. 10/316,137. Similarly, this codrug can be provided in the compositions and devices disclosed in PCT Applications WO 02/87586 and WO 03/024455.
Diclofenac Dimer

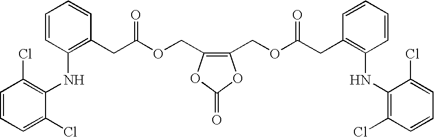

Hydrolysis Rate (half life t½)

| In Buffer, pH7.4 | In Human Plasma |
|---|---|
| 25 hrs | 6 mins |

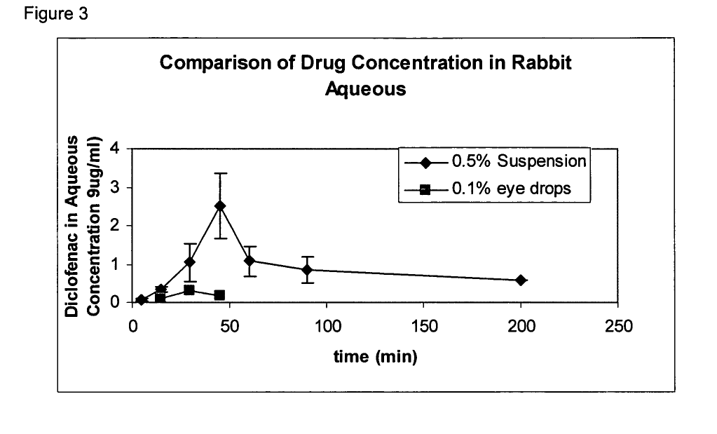

This codrug dimer (or homocodrug) facilitates the preparation of sustained-release formulations of diclofenac. For example, this codrug can be provided in the compositions and devices disclosed in U.S. patent application Ser. No. 10/316,137, and PCT Applications WO 02/87586 and WO 03/024455. A release profile is provided as FIG. 3.
Diclofenac-PEG4

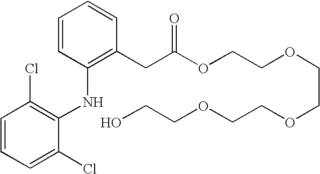

Hydrolysis Rate (half life t½)

| In Buffer, pH7.4 | In Human Plasma |
|---|---|
| 7 days | 10 mins |

This compound can be used as a precursor to a codrug (e.g., using the terminal hydroxl for the attachment of a residue of another drug or prodrug), or can be used itself as a prodrug. This compound has higher water solubility than unionized diclofenac, and is more lipophilic than the ionized form, allowing for formulations that take advantage of this intermediate polarity. This compound is a semi-solid at room temperature and pressure, also facilitating alternative formulations relative to diclofenac or its salts themselves. The compound may be used for the treatment of any disease or condition amenable to treatment by diclofenac itself. This codrug can be provided in the compositions and devices disclosed in U.S. patent application Ser. No. 10/316,137, and PCT Applications WO 02/87586 and WO 03/024455.
Diclofenac-Ofloxacin and Diclofenac-Ciprofloxacin

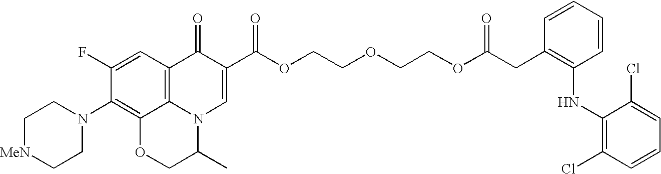

Hydrolysis Rate (half life t½)

| In Buffer, pH7.4 | In Human Plasma |
|---|---|
| days | 6.5 hrs |

This codrug, as well as a codrug salt of diclofenac and ciprofloxacin (i.e., a salt wherein the cation is protonated ciprofloxacin and the anion is deprotonated diclofenac), can be used to treat infections, especially bacterial infections, including those associated with inflammation. The diclofenac-ofloxacin codrug is more lipophilic than ofloxacin alone, and facilitates the preparation of sustained-release formulations. The diclofenac-ciprofloxacin codrug is less water soluble than diclofenac itself, and so is less irritating to local tissues and is easier to formulate for sustained release than diclofenac alone. This codrug also exhibits enhanced permeation. A release profile of the diclofenac-ciprofloxacin codrug is provided as FIG. 2. A comparison between this codrug, formulated as a solution for eyedrops, and commercial ciprofloxacin eyedrops, is provided as FIG. 3, showing the greater duration of release for the codrug. These codrugs can be provided in the compositions and devices disclosed in U.S. patent application Ser. No. 10/316,137, and PCT Applications WO 02/87586 and WO 03/024455.

The foregoing examples and those referenced in the attachment are presented for illustrative purposes only, and are not intended to be limiting. The person skilled in the art will recognize that additional embodiments according to the invention are contemplated as being within the scope of the foregoing generic disclosure, and no disclaimer is in any way intended by the foregoing, non-limiting examples.

All patents, publications, and references cited in the foregoing disclosure are expressly incorporated herein by reference.

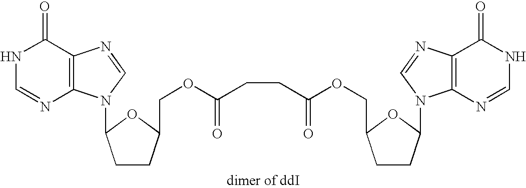

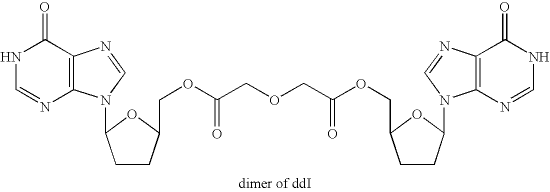

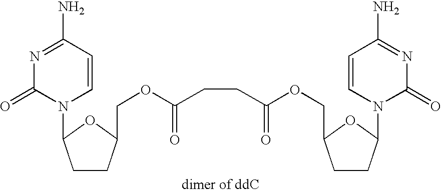

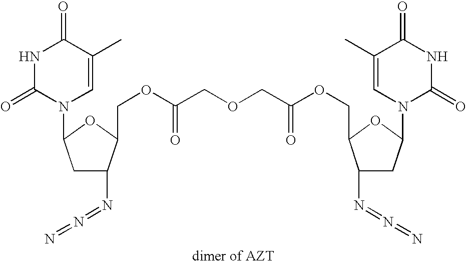

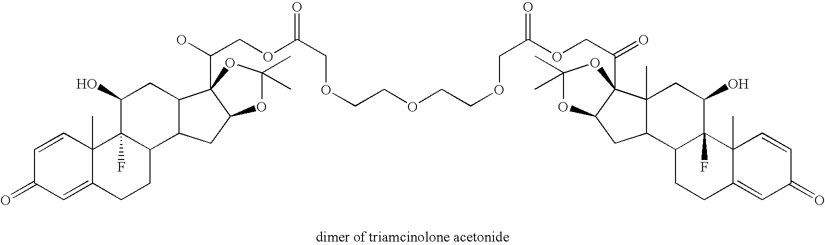

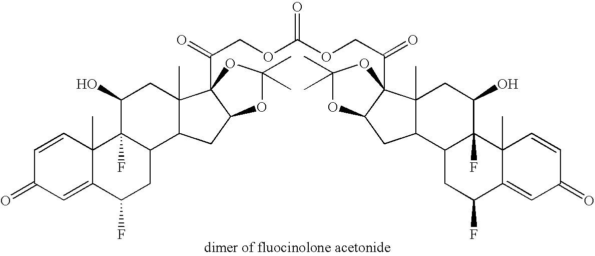

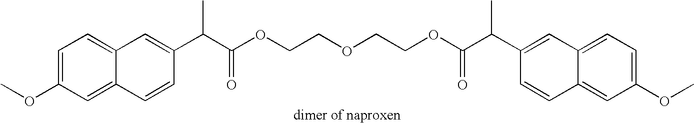

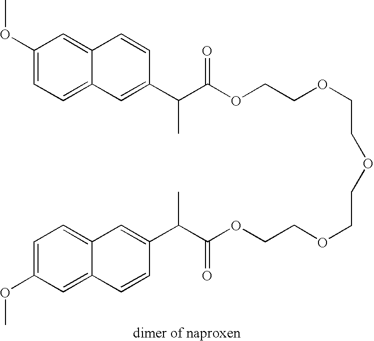

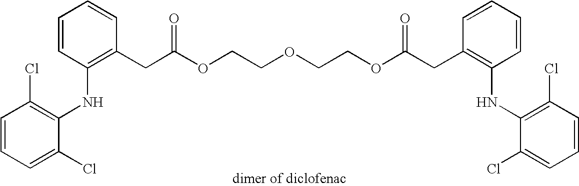

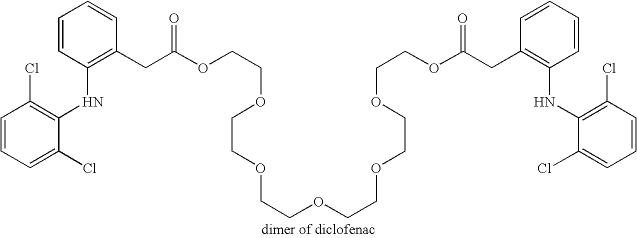

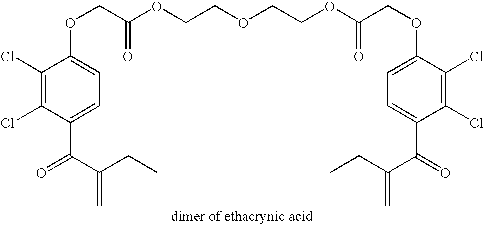

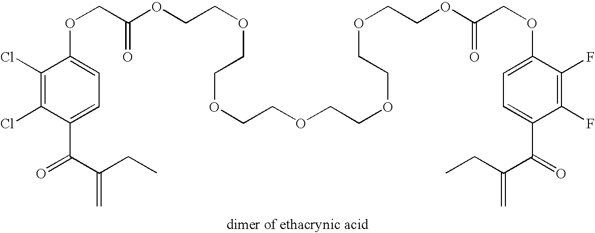

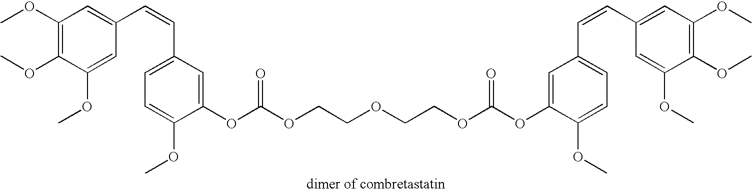

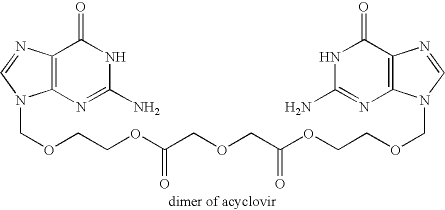

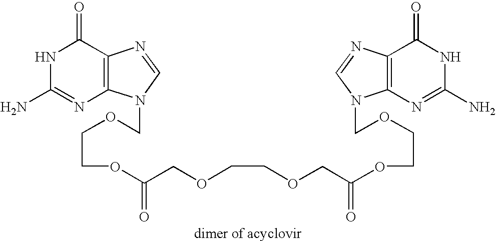

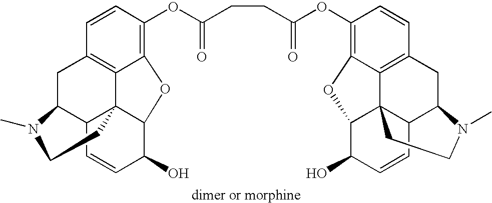

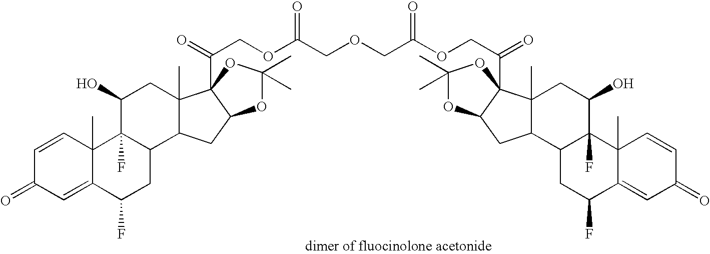

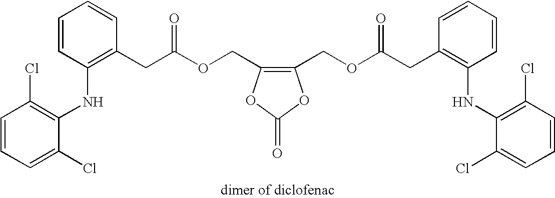

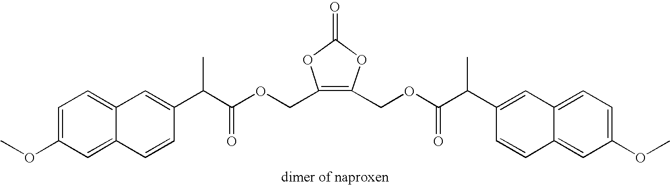

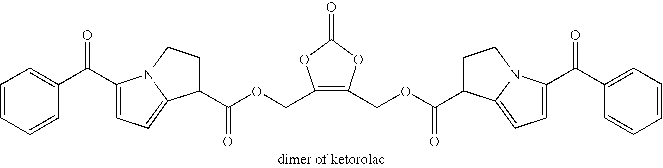

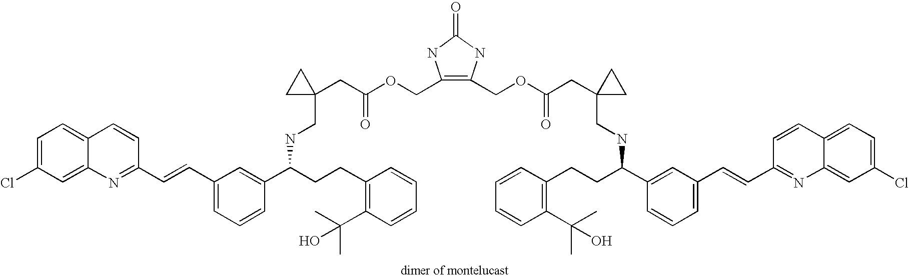

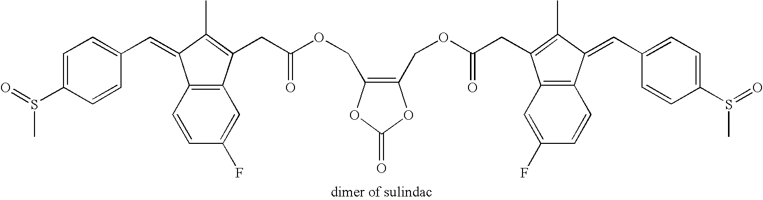

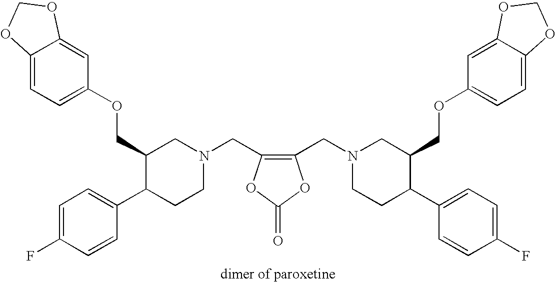

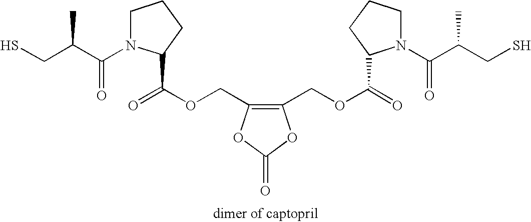

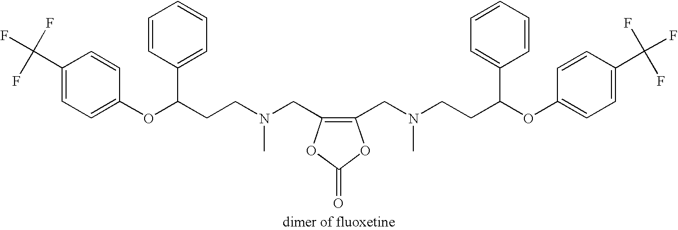

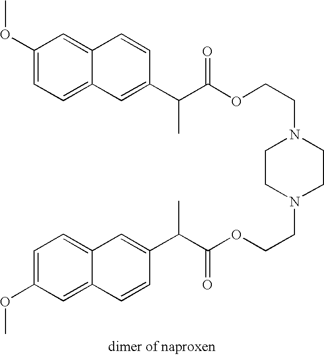

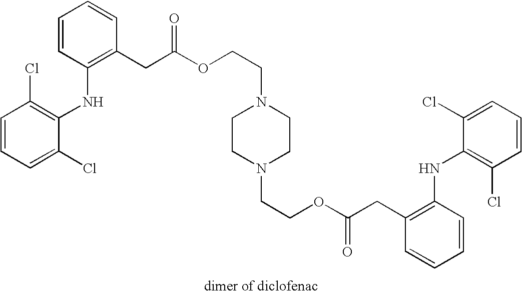

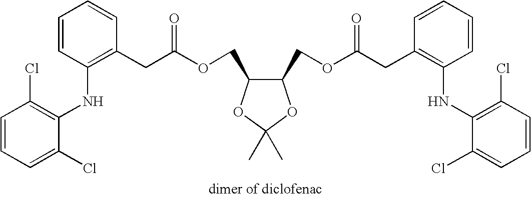

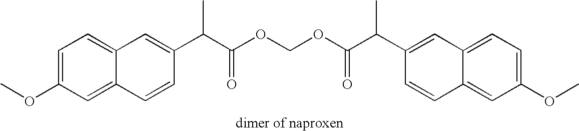

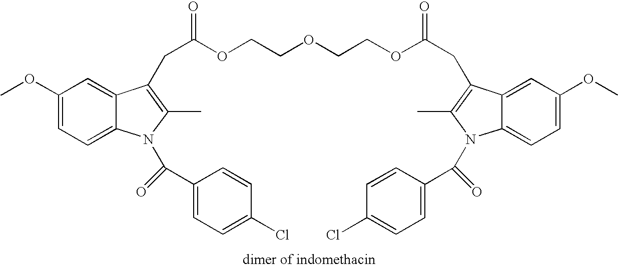

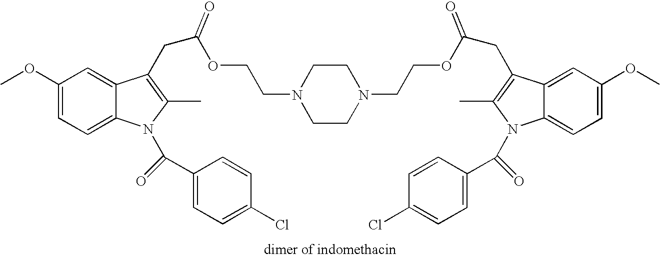

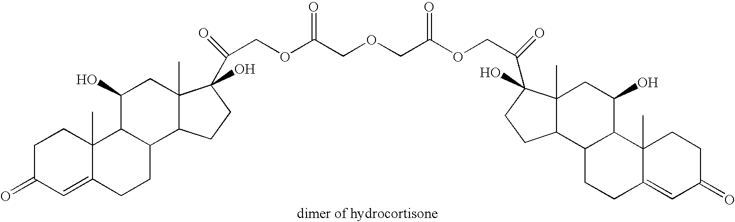

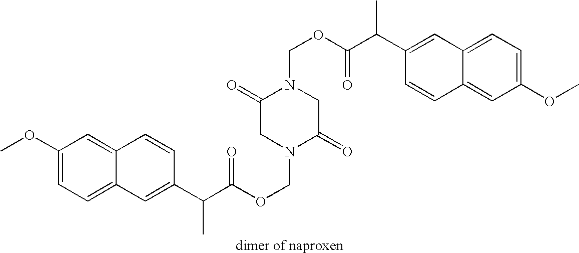

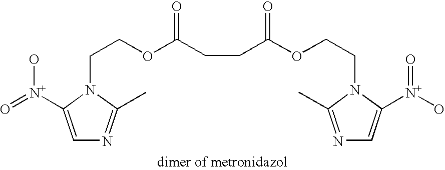

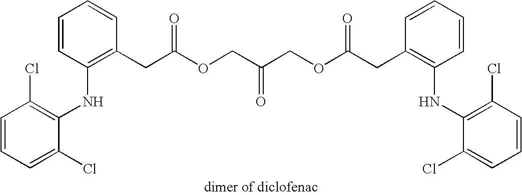

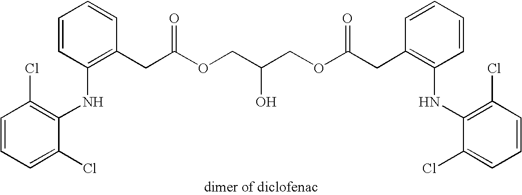

1. A codrug comprising two or more covalently bound moieties, wherein each moiety is a residue of a constituent compound having a biological activity, or a prodrug form thereof, wherein said codrug is cleaved under physiological conditions to regenerate each constituent compound, and wherein at least one moiety has a decomposition rate that is at least 10% less when stored at ambient temperature in codrug form than when stored as an unlinked compound.

2. A codrug comprising two or more covalently bound moieties, wherein each moiety is a residue of a constituent compound having a biological activity, or a prodrug form thereof, wherein said codrug is cleaved under physiological conditions to regenerate each constituent compound, and wherein the codrug is at least 1 0% more soluble than at least one of the unlinked constituent compounds in a polymeric delivery system.

3. A codrug comprising two or more covalently bound moieties, wherein each moiety is a residue of a constituent compound having a biological activity, or a prodrug form thereof, wherein said codrug is cleaved under physiological conditions to regenerate each constituent compound, and wherein the codrug is more readily formulated as a powder than is at least one of the constituent compounds when existing in unlinked form.

4. A codrug comprising two or more covalently bound moieties, wherein each moiety is a residue of a constituent compound having a biological activity, or a prodrug form thereof, wherein said codrug is cleaved under physiological conditions to regenerate each constituent compound, and wherein the codrug is at least 10% more soluble in a pharmaceutically acceptable carrier than is at least one of the unlinked constituent compounds.

5. A codrug comprising two or more covalently bound moieties, wherein each moiety is a residue of a constituent compound having a biological activity, or a prodrug form thereof, wherein said codrug is cleaved under physiological conditions to regenerate each constituent compound, and wherein the codrug is more readily formulated in solid dosage forms than is at least one of the constituent compounds when existing in unlinked form.

6. A codrug comprising two or more covalently bound moieties, wherein each moiety is a residue of a constituent compound having a biological activity, or a prodrug form thereof, wherein said codrug is cleaved under physiological conditions to regenerate each constituent compound, and wherein the codrug is at least 10% more soluble in organic solvents than is at least one of the unlinked constituent compounds.

7. A codrug comprising two or more covalently bound moieties, wherein each moiety is a residue of a constituent compound having a biological activity, or a prodrug form thereof, wherein said codrug is cleaved under physiological conditions to regenerate each constituent compound, and wherein each moiety exhibits a release profile that is comparable to the release profile of the constituent compounds.

8. A codrug comprising two or more covalently bound moieties, wherein each moiety is a residue of a constituent compound having a biological activity, or a prodrug form thereof, wherein said codrug is cleaved under physiological conditions to regenerate each constituent compound, and wherein the codrug is at least 10% more soluble in physiological fluids than is at least one of the unlinked constituent compounds.

9. A codrug comprising two or more covalently bound moieties, wherein each moiety is a residue of a constituent compound having a biological activity, or a prodrug form thereof, wherein said codrug is cleaved under physiological conditions to regenerate each constituent compound, and wherein the codrug is at least 10% more soluble at a pH greater than 7.4 at 37° C. than is at least one of the unlinked constituent compounds.

10. A codrug comprising two or more covalently bound moieties, wherein each moiety is a residue of a constituent compound having a biological activity, or a prodrug form thereof, wherein said codrug is cleaved under physiological conditions to regenerate each constituent compound, and wherein the codrug is at least 10% more soluble at a pH less than 7.4 at 37° C. than is at least one of the unlinked constituent compounds.

11. A codrug comprising two or more covalently bound moieties, wherein each moiety is a residue of a constituent compound having a biological activity, or a prodrug form thereof, wherein said codrug is cleaved under physiological conditions to regenerate each constituent compound, and wherein the codrug is at least 10% more stable at a pH less than 7.4 at 37° C. than is at least one of the unlinked constituent compounds.

12. A codrug comprising two or more covalently bound moieties, wherein each moiety is a residue of a constituent compound having a biological activity, or a prodrug form thereof, wherein said codrug is cleaved under physiological conditions to regenerate each constituent compound, and wherein the codrug is at least 10% more stable at a pH greater than 7.4 at 37° C. than is at least one of the unlinked constituent compounds.

13. A dosage form comprising a codrug wherein the codrug comprises two or more covalently bound moieties, wherein each moiety is a residue of a constituent compound having a biological activity, or a prodrug form thereof, wherein said codrug is cleaved under physiological conditions to regenerate each constituent compound, and wherein each moiety exhibits a release profile that is comparable to the release profile of the constituent compounds.

14. A codrug of any of claims 1 through 12, wherein the codrug is represented by the formula A₁(—A₂)ₙ.

15. A codrug of any of claims 1 through 12, wherein the codrug is represented by the formula A₁(—L—A₂)ₙ, wherein L is a linking group.

16. A composition comprising the codrug of any of claims 1 through 12, wherein the composition is substantially pyrogen-free.

17. A composition comprising the codrug of any of claims 1 through 12 and a pharmaceutically acceptable carrier, diluent, adjuvant, or excipient.

18. A bioerodible drug delivery device comprising the codrugs of any of claims 1 through 12, wherein the device delivers at least one of the constituent compounds at a therapeutically effective dose over a period of at least three hours.

19. A non-bioerodible drug delivery device comprising the codrugs of any of claims 1 through 12, wherein the device delivers at least one of the constituent compounds at a therapeutically effective dose over a period of at least two days.

20. A method for administering a compound to a patient, comprising administering the codrugs of any of claims 1 through 12 to a patient in need thereof.

21. A method for administering a therapeutically effective compound to a patient, comprising administering the codrugs of any of claims 1 through 12 to a patient in need thereof, wherein said codrug or composition is provided in a bioerodible drug delivery device that delivers the compound over a period of at least two days.

22. A method for administering a compound to a patient, comprising administering the codrugs of any of claims 1 through 12 to a patient in need thereof, wherein said codrug or composition is provided in a non-bioerodible drug delivery device that delivers the compound at a therapeutically effective dose over a period of at least two days.

23. A method for administering a compound to a patient, comprising administering the codrug of claims 1 through 12, or the composition of claim 17 to a patient in need thereof, wherein the compound is delivered at a therapeutically effective dose to a localized area within a body while maintaining a therapeutically ineffective systemic concentration of said codrug within said body as a whole.

24. A method of making a codrug of any of claims 1 through 12 comprising:
combining a precursor of the first moiety with a precursor of the second moiety through a covalent linkage to form a precursor codrug, wherein the first moiety has at least one reactive group and the covalent linkage occurs through the reactive group, and
converting the precursor codrug to the codrug by subjecting the precursor codrug to at least 1 reaction.

25. A codrug of any of claims 1 through 12, wherein said codrug is synthesized by:
combining a precursor of the first moiety with a precursor of the second moiety through a covalent linkage to form a precursor codrug, wherein the first moiety has at least one reactive group and the covalent linkage occurs through the reactive group, and
converting the precursor codrug to the codrug by subjecting the precursor codrug to at least 1 reaction.

26. A method of making a codrug of any of claims 1 through 12 comprising:
combining a precursor of the first moiety with the second moiety through a covalent linkage to form a precursor codrug, wherein the first moiety has at least one reactive group, the covalent linkage occurs through the reactive group, and the second moiety is not in precursor form, and
converting the precursor codrug to the codrug by subjecting the precursor codrug to at least 1 reaction.

27. A codrug of any of claims 1 through 12, wherein said codrug is synthesized by:
combining a precursor of the first moiety with the second moiety through a covalent linkage to form a precursor codrug, wherein the first moiety has at least one reactive group, the covalent linkage occurs through the reactive group, and the second moiety is not in precursor form, and
converting the precursor codrug to the codrug by subjecting the precursor codrug to at least 1 reaction.

28. A formulation comprising a codrug comprising two or more covalently bound moieties, wherein each moiety is a residue of a constituent compound having a biological activity, or a prodrug form thereof, wherein the codrug has improved in vivo stability compared to the in vivo stability of at least one of the constituent compounds.

29. A method for administering a therapeutically effective compound to a patient, comprising orally administering a codrug of any of claims 1 through 12 to a patient in need thereof.

30. A method for administering a therapeutically effective compound to a patient, comprising systemically administering a codrug of any of claims 1 through 12 to a patient in need thereof in a therapeutically effective dose.

31. A compound having a structure selected from:

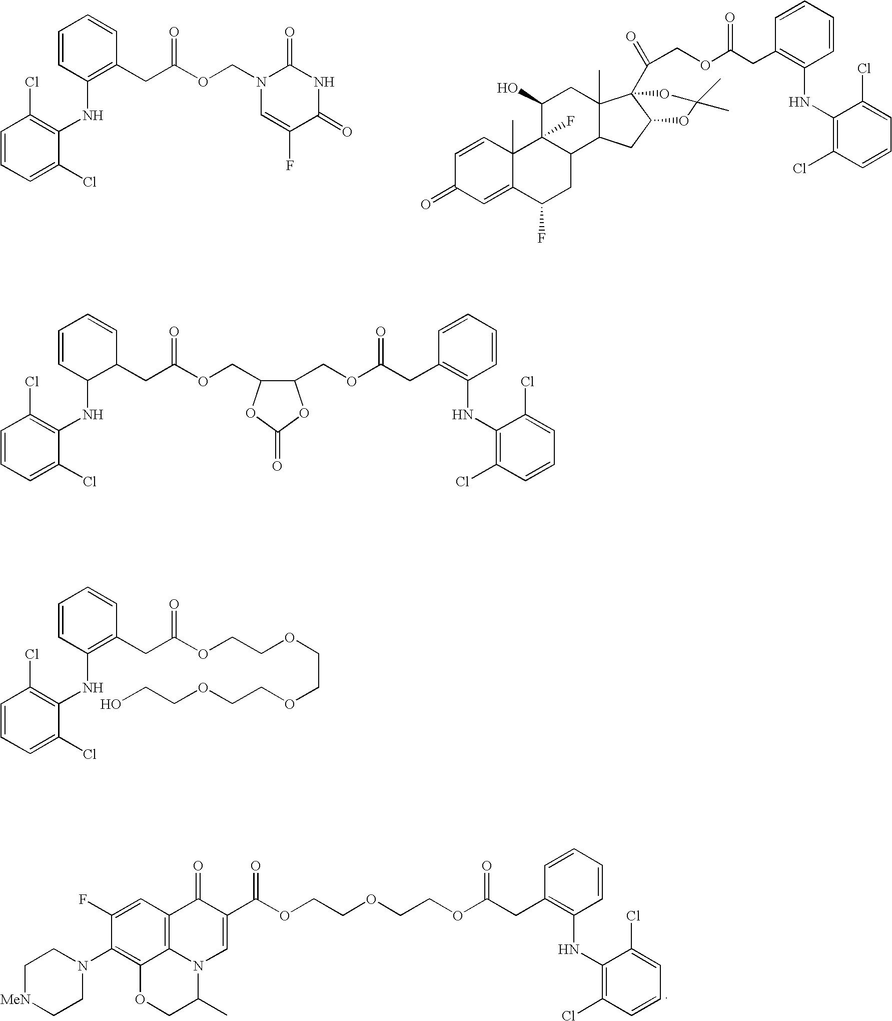

32. A salt of ciprofloxacin with diclofenac.
